(19) United States
(12) Patent Application Publication
(10) Pub. No.: US 2022/0381779 A1
(43) Pub. Date: Dec. 1, 2022

(54) METHODS FOR THE DIAGNOSIS OF BURULI ULCER

(71) Applicants: INSERM (INSTITUT NATIONAL DE LA SANTÉ ET DE LA RECHERCHE MÉDICALE), Paris (FR); UNIVERSITÉ D' ANGERS, Angers (FR)

(72) Inventors: Laurent MARSOLLIER, Angers (FR); Estelle MARION, Angers (FR); Mélanie FOULON, Angers (FR)

(21) Appl. No.: 17/771,103

(22) PCT Filed: Apr. 11, 2020
(86) PCT No.: PCT/EP2020/080923

§ 371 Date: Apr. 22, 2022

(30) Foreign Application Priority Data
May 11, 2019 (EP) 19207074.6

(51) Int. Cl. G01N 33/569

(52) U.S. Cl. CPC G01N 33/5695; G01N 2469/20; G01N 2333/35

(57) ABSTRACT

The present invention relates to the diagnosis of Buruli ulcer. The inventors investigated the potential role of local natural antibodies in the recognition of mycolactone produced by M. ulcerans, and confirmed the presence of such antibodies in humans. In particular, the present invention relates to a method of diagnosing Buruli ulcer in a subject comprising detecting anti-mycolactone immunoglobulin (anti-mycolactone IgG) in a biological sample obtained from said subject.

### FIELD OF THE INVENTION

The present invention relates to the diagnosis of Buruli ulcer.

### BACKGROUND OF THE INVENTION

Buruli ulcer, a neglected tropical disease caused by Mycobacterium ulcerans, is the third most common mycobacterial disease in the world after tuberculosis and leprosy (1). This chronic infectious disease is characterized by the destruction of cutaneous tissue, leading to the development of large ulcerative lesions. This tissue destruction is caused by a unique lipid-like toxin called mycolactone, produced by M. ulcerans. Mycolactone is cytotoxic at high doses, but, at lower doses, it modulates pain and immune responses, facilitating host colonization (2, 3, 4, 5, 6). Despite this toxin secretion, experimental approaches, supported by observations in human patients, have revealed that the modulation of the immune response induced by mycolactone is local and regional, but not systemic (7, 8). Moreover, the systemic humoral response to M. ulcerans infection is weak (8) and M. ulcerans is poorly recognized by circulating antibodies (9). Histological analyses of patient tissues have shown that Buruli ulcer lesions are surrounded by a massive inflammatory infiltrate of leukocytes (10). This local infiltrate has been characterized in mice and has been shown to consist predominantly of phagocytes (mostly macrophages and neutrophils) and lymphocytes (8). Overall, these findings suggest that the immune response cannot control lesion development, despite the recruitment of several major actors of the immune system. This failure of host immunity is a consequence of the strategy used by M. ulcerans to escape to the immune system.

The previous studies were performed during the pre-ulcerative and ulcerative phases, but no study to date has considered these aspects during the spontaneous healing stage. Buruli ulcer can be treated with antibiotics during early stages. But, despite improvement in treatments, about 25% of Buruli ulcer patients become permanently disabled (11). Furthermore, untreated lesions can spread to entire limbs and progress to chronic skin ulcers (12). However, in the absence of medical treatment, spontaneous healing occurs in 5% of cases, as recently reported in Africa and confirmed by the spontaneous healing of untreated Australian patients with M. ulcerans infections (13, 14), suggesting the possible establishment of a strategy counteracting the effects of M. ulcerans in the host. This observation reveals that the host plays a role in controlling lesion development. A recent histological study showed that B cells accumulate in clusters around M. ulcerans infection sites, suggesting a specific local adaptive response (10). Indeed, B cells are known to produce immunoglobulin, and constitute the humoral arm of the immune system. These lymphocytes have both pro-inflammatory and suppressive roles in the pathophysiology of inflammatory skin disorders (15). Furthermore, skin-associated B cells have been shown to be different from lymph node B cells (16). Finally, this specific local humoral response may enhance local defense and immunity in the context of chronic inflammatory skin diseases (17).

However, the local humoral response has never been investigated during the course of M. ulcerans infection, including the spontaneous healing phase. Using an original mouse model reproducing the key features of the disease in humans (including spontaneous healing after the ulcerative phase, as previously described by Marion et al (8), the inventors investigated the potential role of local natural antibodies in the recognition of mycolactone produced by M. ulcerans, and confirmed the presence of such antibodies in humans.

Therefore, there is a need for new biological marker of Buruli ulcer. There is currently no simple diagnostic tool suitable for use in the rural areas of developing countries. This situation is particularly regrettable, as the early stages of Buruli ulcer can be treated locally, whereas the treatment of later stages requires extensive surgery in larger hospitals, with longer periods of hospitalization, at much greater expense.

### SUMMARY OF THE INVENTION

The present invention relates to methods and kits for diagnosing Buruli ulcer. In particular, the present invention is defined by the claims.

### DETAILED DESCRIPTION OF THE INVENTION

Buruli ulcer, a neglected tropical disease, is a chronic infectious disease caused by Mycobacterium ulcerans. Without treatment, lesions caused by the M. ulcerans main virulence factor, mycolactone, can escalate into chronic skin ulcers. Spontaneous healing of these severe lesions was observed in 5% of patients' cases suggesting the possible establishment of a strategy counteracting the effects of M. ulcerans in the host. Using our mice model of spontaneous healing, we revealed the role of the host machinery in controlling lesion development through the identification of a skin-specific local humoral signature of the spontaneous healing process. We highlighted for the first time the production of skin-specific antibodies neutralizing the activity of the mycolactone toxin. The potential role of human host machinery in triggering effective local immune response was further confirmed in Buruli ulcer patients and supported by molecular profiling. The inventor's findings pave the way for significant advances in both the diagnosis of Buruli ulcer by means of bioassays based on monoclonal antibody production and the development of novel treatment and vaccine by mean of mycolactone-neutralizing strategies. These perspectives are in perfect line with the most recent challenges issued by the World Health Organization.

Accordingly, a first object of the present invention relates to a method of diagnosing Buruli ulcer in a subject comprising detecting immunoglobulin able to recognize M. ulcerans components in a biological sample obtained from said subject.

In particular, the present invention relates to a method of diagnosing Buruli ulcer in a subject, comprising detecting immunoglobulin able to recognize mycolactone in a biological sample obtained from said subject.

In particular, the present invention relates to a method of diagnosing Buruli ulcer in a subject, comprising detecting anti-mycolactone immunoglobulin in a biological sample obtained from said subject.

As used herein, the term “subject” denotes a mammal, such as a rodent, a feline, a canine, and a primate. Particularly, the subject according to the invention is a human. More particularly, the subject according to the invention has or is susceptible to have Buruli ulcer. As used herein, the term “subject” encompasses “patient”.

As used herein, the term “biological sample” refers to any sample obtained from a subject, such as a skin tissue, a serum sample, a plasma sample, a urine sample, a blood sample, a lymph sample, or a tissue biopsy. In a particular embodiment, the biological sample is a tissue biopsy. Particularly, the biological sample is a skin tissue.

As used herein, the term “tissue”, when used in reference to a part of a body or of an organ, generally refers to an aggregation or collection of morphologically similar cells and associated accessory and support cells and intercellular matter, including extracellular matrix material, vascular supply, and fluids, acting together to perform specific functions in the body. There are generally four basic types of tissue in animals and humans including muscle, nerve, epithelial, and connective tissues.

As used herein, the term “Buruli ulcer” has its general meaning in the art and refers to an infectious disease caused by Mycobacterium ulcerans. The early stage of the infection is characterised by a painless nodule or area of swelling. This nodule can turn into an ulcer. The ulcer may be larger inside than at the surface of the skin, and can be surrounded by swelling. As the disease worsens, bone can be infected. Buruli ulcers most commonly affect the arms or legs; fever is uncommon.

In some embodiments, the method of diagnosing described herein is applied to an infected subject who presents symptoms of Buruli ulcer. The different symptoms of Buruli ulcer are, gradually, the following: redness, edema, ulcer, necrosis.

In particular, the anti-mycolactone immunoglobulin is detected in all stages of infection.

As used herein, the term “healthy” has its general meaning in the art and refers to a subject in a good physical and mental condition, in good health.

As used herein, the term “infected” has its general meaning in the art and refers to as subject affected with a disease-causing organism.

As used herein, the term “redness” has its general meaning in the art and refers to the quality or state of a skin portion being red or reddish.

As used herein, the term “edema” has its general meaning in the art and refers to an abnormal accumulation of fluid in the interstitium, located beneath the skin and in the cavities of the body, which can cause severe pain. Clinically, hyperaldosteronism, edema manifests as swelling.

As used herein, the term “ulcers” has its general meaning in the art and refers to a discontinuity or break in a bodily membrane that impedes the organ of which that membrane is a part from continuing its normal functions.

As used herein, the term “necrosis” has its general meaning in the art and results in the premature death of cells in living tissue by autolysis. Necrosis is caused by factors external to the cell or tissue, such as infection, toxins, or trauma which result in the unregulated digestion of cell components.

As used herein, the term “Mycobacterium ulcerans” (M. ulcerans) has its general meaning in the art and refers to a slow-growing Mycobacterium that classically infects the skin and subcutaneous tissues, giving rise to indolent nonulcerated (nodules, plaques) and ulcerated lesions.

As used herein, the term “mycolactone” also known as “[(6S,7S,9E,12R)-12-[(E,2S,6R,7R,9R)-7,9-dihydroxy-4,6-dimethyldec-4-en-2-yl]-7,9-dimethyl-2-oxo-1-oxacyclododec-9-en-6-yl] (2E,4E,6E,8E,10E,12S,13S,15S)-12,13,15-trihydroxy-4,6,10-trimethylhexadeca-2,4,6,8,10-pentaenoate” has its general meaning in the art and refers to a polyketide-derived macrolide produced and secreted by a group of very closely related pathogenic Mycobacteria species. In a particular embodiment, the mycolactone is a compound of formula:

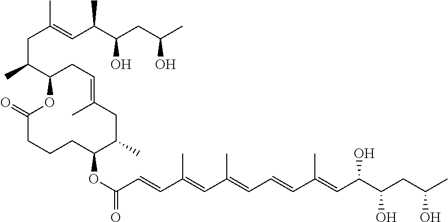 [SMILES: CC(=C\C=C\C(C)=C\[C@H](O)[C@@H](O)C[C@H](C)O)/C=C(C)/C=C/C(=O)O[C@H]1CCCC(=O)O[C@@H]([C@@H](C)C/C(C)=C/[C@@H](C)[C@H](O)C[C@@H](C)O)C/C=C(\C)C[C@@H]1C]

As used herein, the term “control sample” refers to a skin tissue or cells from a subject non-diagnosed as Buruli ulcer patients.

As used herein, the term “infected sample” refers to a skin tissue or cells from an infected subject (displaying redness, edema, ulcer, necrosis), or to an infected tissue of the subject (displaying redness, edema, ulcer necrosis).

As used herein the term “immunoglobulin” or “antibody” have the same meaning, and will be used equally in the present invention. The term “antibody” as used herein refers to immunoglobulin molecules and immunologically active portions of immunoglobulin molecules, i.e., molecules that contain an antigen binding site that immunospecifically binds an antigen. As such, the term antibody encompasses not only whole antibody molecules, but also antibody fragments as well as variants (including derivatives) of antibodies and antibody fragments, and fusion protein comprising an antigen-binding portion of an antibody. In natural antibodies, two heavy chains are linked to each other by disulfide bonds and each heavy chain is linked to a light chain by a disulfide bond. There are two types of light chain, lambda (1) and kappa (κ). There are five main heavy chain classes (or isotypes) which determine the functional activity of an antibody molecule: IgM, IgD, IgG, IgA and IgE. Each chain contains distinct sequence domains. The light chain includes two domains, a variable domain (VL) and a constant domain (CL). The heavy chain includes four domains, a variable domain (VH) and three constant domains (CHI, CH2 and CH3, collectively referred to as CH). The variable regions of both light (VL) and heavy (VH) chains determine binding recognition and specificity to the antigen. The constant region domains of the light (CL) and heavy (CH) chains confer important biological properties such as antibody chain association, secretion, trans-placental mobility, complement binding, and binding to Fc receptors (FcR). The Fv fragment is the N-terminal part of the Fab fragment of an immunoglobulin and consists of the variable portions of one light chain and one heavy chain. The specificity of the antibody resides in the structural complementarity between the antibody combining site and the antigenic determinant. Antibody combining sites are made up of residues that are primarily from the hypervariable or complementarity determining regions (CDRs). Occasionally, residues from nonhypervariable or framework regions (FR) can participate to the antibody binding site or influence the overall domain structure and hence the combining site. Complementarity Determining Regions or CDRs refer to amino acid sequences which together define the binding affinity and specificity of the natural Fv region of a native immunoglobulin binding site. The light and heavy chains of an immunoglobulin each have three CDRs, designated L-CDR1, L-CDR2, L-CDR3 and H-CDR1, H-CDR2, H-CDR3, respectively. An antigen-binding site, therefore, typically includes six CDRs, comprising the CDR set from each of a heavy and a light chain V region. Framework Regions (FRs) refer to amino acid sequences interposed between CDRs. Each VH and VL is composed of three CDRs and four FRs arranged from amino-terminus to carboxy-terminus in the following order: FR1, CDR1, FR2, CDR2, FR3, CDR3, FR4.

Furthermore, although the two domains of the Fv fragment, VL and VH, are coded for by separate genes, they can be joined, using recombinant methods, by a synthetic linker that enables them to be made as a single chain protein in which the VL and VH regions pair to form monovalent molecules (known as single chain Fv (scFv); see e.g., Bird et al., 1988 Science 242:423-426; and Huston et al., 1988 Proc. Natl. Acad. Sci. 85:5879-5883). Such single chain antibodies are also intended to be encompassed within the term “antigen-binding portion” of an antibody. These antibody fragments are obtained using conventional techniques known to those of skill in the art, and the fragments are screened for utility in the same manner as are intact antibodies.

An “isolated antibody”, as used herein, refers to an antibody that is substantially free of other antibodies having different antigenic specificities (e.g., an isolated antibody that specifically binds to a conformational epitope of GP is substantially free of antibodies that specifically bind to other distinct epitopes of GP or epitopes on distinct proteins). An isolated antibody that specifically binds to a conformational epitope of GP may, however, have cross-reactivity to other antigens, such as similar conformational epitopes of GP proteins from other Ebolavirus species. Moreover, an isolated antibody may be substantially free of other cellular material and/or chemicals.

The term “human antibody”, as used herein, is intended to include antibodies having variable regions in which both the framework and CDR regions are derived from sequences of human origin. Furthermore, if the antibody contains a constant region, the constant region also is derived from such human sequences, e.g., human germline sequences, or mutant versions of human germline sequences or antibody containing consensus framework sequences derived from human framework sequences analysis.

As use herein, the term “IgG” means a class of immunoglobulins including the most common antibodies circulating in the blood that facilitate the phagocytic destruction of microorganisms foreign to the body, that bind to and activate complement, and that are the only immunoglobulins to cross over the placenta from mother to fetus. Immunoglobulin G (IgG) is an antibody isotype. It is a protein complex composed of four peptide chains-two identical heavy chains and two identical light chains arranged in a Y-shape typical of antibody monomers. Each IgG has two antigen binding sites. Representing approximately 75% of serum immunoglobulins in humans, IgG is the most abundant antibody isotype found in the circulation. IgG molecules are synthesized and secreted by plasma B cells. There are four IgG subclasses (IgG 1, 2, 3, and 4) in humans, named in order of their abundance in serum (IgG1 being the most abundant).

As use herein the term “anti-mycolactone immunoglobulin” (anti-mycolactone IgG) refers to the immunoglobulins (i.e. antibodies) which are produced by the immune system of the subject and that are directed against mycolactone.

In one embodiment, the method of the invention is performed to detect subject with anti-mycolactone IgG.

In a preferred embodiment, the subject is having or at risk of having or developing Buruli ulcer, if the detection of anti-mycolactone IgG is positive (i.e. superior to zero).

In a preferred embodiment, the subject is not having or at risk of having or developing Buruli ulcer, if the detection of anti-mycolactone IgG is negative (i.e. inferior to zero).

The detection and quantification of anti-mycolactone IgG in the sample can be detected by any method known in the art. Typically the detection and quantification is performed by Enzyme-linked immunosorbent assay, also called ELISA, enzyme immunoassay or EIA, is a biochemical technique used mainly in immunology to detect the presence of an antibody or an antigen in a sample. A known amount of mycolactone is immobilized on a solid support (usually a polystyrene micro titer plate) either non-specifically (via adsorption to the surface) or specifically (via capture by another antibody specific to the same antigen, in a “sandwich” ELISA). Then the sample, suspected of containing anti-mycolactone IgG, is washed over the surface so that the antibodies can bind to the immobilized antigen. The surface is washed to remove any unbound protein and a detection antibody is applied to the surface. The detection antibody should be an anti-human IgG antibody. The detection antibody can be covalently linked to an enzyme, or can itself be detected by a secondary antibody which is linked to an enzyme through bio-conjugation. Enzymes which can be used to detectably label the antibodies of the present invention include, but are not limited to, malate dehydrogenase, staphylococcal nuclease, delta-V-steroid isomerase, yeast alcohol dehydrogenase, alpha-glycerophosphate dehydrogenase, triose phosphate isomerase, horseradish peroxidase, alkaline phosphatase, asparaginase, glucose oxidase, beta-galactosidase, ribonuclease, urease, catalase, glucose-VI-phosphate dehydrogenase, glucoamylase and acetylcholinesterase. Between each step the plate is typically washed with a mild detergent solution to remove any proteins or antibodies that are not specifically bound. After the final wash step the plate is developed by adding an enzymatic substrate to produce a visible signal. In some embodiments, a competitive ELISA is used.

Purified anti-mycolactone antibodies that are not derived from the subject are coated on the solid phase of multi-wells. Serum sample recombined anti-mycolactone IgG, (the antigen) or fragments thereof and horseradish peroxidase labelled with anti-mycolactone antibodies (conjugated) are added to coated wells, and form competitive combination. After incubation, if the antibody level against mycolactone content is high in the sample, a complex of mycolactone-anti-mycolactone antibodies labelled with HRP will form. Washing wells will remove the complex, and incubation with TMB (3,3′,5,5′-tetramethylbenzidene) will lead to the color development substrate for localization of horseradish peroxidase-conjugated antibodies in the wells. Subsequently there will be no color change or little color change. If there is no anti-mycolactone IgG in the serum sample, there will be much color change. Such a competitive ELSA test is specific, sensitive, reproducible and easy to operate. In some embodiments, the detection antibody is label with a fluorescent compound. When the fluorescently labelled antibody is exposed to light of the proper wavelength, its presence can then be detected due to fluorescence. Among the most commonly used fluorescent labeling compounds are CY dyes, fluorescein isothiocyanate, rhodamine, phycoerytherin, phycocyanin, allophycocyanin, o-phthaldehyde and fluorescamine. In some embodiments the detection antibody can also be detectably labeled using fluorescence emitting metals such as 152Eu, or others of the lanthanide series. These metals can be attached to the antibody using such metal chelating groups as diethylenetriaminepentaacetic acid (DTPA) or ethylenediaminetetraacetic acid (EDTA). In some embodiments, the detection antibody is detectably labeled by coupling it to a chemiluminescent compound. The presence of the chemiluminescent-antibody is then determined by detecting the presence of luminescence that arises during the course of a chemical reaction. Examples of particularly useful chemiluminescent labeling compounds are luminol, luciferin, isoluminol, theromatic acridinium ester, imidazole, acridinium salt and oxalate ester. There are other different forms of ELISA, which are well known to those skilled in the art. In some embodiments, the immunoassays comprise beads coated with native or recombinant mycolactone as described. Commonly used are polystyrene beads that are dyed to establish a unique identity. Detection is performed by flow cytometry. Other types of bead-based immunoassays are well known in the art, e.g. laser bead immunoassays and related magnetic bead assays (Fritzler, Marvin J; Fritzler, Mark L, Expert Opinion on Medical Diagnostics, 2009, pp. 3: 81-89).

A further object of the present invention relates to a kit or device for identifying the presence or the concentration of anti-mycolactone IgG in a sample from a subject comprising: at least a mycolactone or fragments thereof; and at least one solid support wherein the mycolactone or fragments thereof is deposited on the support. In some embodiments, the mycolactone or fragments thereof that is deposited on the solid support is immobilized on the support. In some embodiments, the solid support is in the format of a dipstick, a test strip, a latex bead, a microsphere or a multi-well plate. In some embodiments, the devices or kits described herein can further comprise a second labelled mycolactone or a fragment thereof which produces a detectable signal; a detection antibody, wherein the detection antibody is specific for the anti-mycolactone IgG in the sample of the subject and the detection antibody produces a detectable signal; or a nephelometer cuvette. In some embodiments, the device performs an immunoassay wherein an antibody-protein complex is formed, such as a serological immunoassay or a nephelometric immunoassay. In some embodiments, provided herein are kits that comprise devices described herein and a detection antibody, wherein the detection antibody is specific for the anti-mycolactone IgG in the sample of the subject and produces a detectable signal. In some embodiments, the kit can include a second labelled mycolactone protein or a fragment thereof which produces a detectable signal. In some embodiments, the kit includes a nephelometer cuvette. Any solid support can be used, including but not limited to, nitrocellulose membrane, nylon membrane, solid organic polymers, such as polystyrene, or laminated dipsticks such as described in U.S. Pat. No. 5,550,375. The use of “dip sticks” or test strips and other solid supports have been described in the art in the context of an immunoassay for a number of antigens. Three U.S. patents (U.S. Pat. No. 4,444,880, issued to H. Tom; U.S. Pat. No. 4,305,924, issued to R. N. Piasio; and U.S. Pat. No. 4,135,884, issued to J. T. Shen) describe the use of “dip stick” technology to detect soluble antigens via immunochemical assays. The apparatuses and methods of these three patents broadly describe a first component fixed to a solid surface on a “dip stick” which is exposed to a solution containing a soluble antigen that binds to the component fixed upon the “dip stick,” prior to detection of the component-antigen complex upon the stick. The “dip stick” technology can be easily adapted for the present invention by one skilled in the art. In the invention described herein, the mycolactone is deposited on the support and the auto-antibody is to be detected. Examples of kits include but are not limited to ELISA assay kits, and kits comprising test strips and dipsticks. In an ELISA kit, an excess amount of mycolactone is immobilized on a solid support. A sample containing an unknown amount of anti-mycolactone IgG is added to the immobilized mycolactone, resulting in the formation of a complex consisting of the antigen and the antibody. The complex is detected by a labelled secondary antibody that is also specific for the antibody. In some embodiments of the kits described herein, the kit comprises a test strip or a dipstick. In a typical colloidal gold labelling technique, the unique red color of the accumulated gold label, when observed by lateral or transverse flow along a membrane on which an antigen is captured by an immobilized antibody, or by observation of the red color intensity in solution, provides an extremely sensitive method for detecting sub nanogram quantities of proteins (or antigens) in solution. A colloidal gold conjugate consists of a suspension of gold particles coated with a selected protein or macromolecule (such as an antibody or antibody-based moiety). The gold particles may be manufactured to any chosen size from 1-250 nm. This gold probe detection system, when incubated with a specific target, such as in a tissue section, will reveal the target through the visibility of the gold particles themselves. For detection by eye, gold particles will also reveal immobilized antigen on a solid phase such as a blotting membrane through the accumulated red color of the gold sol. Silver enhancement of this gold precipitate also gives further sensitivity of detection. Suppliers of colloidal gold reagents for labelling are available from SPI-MARK™. Polystyrene latex Bead size 200 nm colored latex bead coated with antibody SIGMA ALDRICH®, Molecular Probes, Bangs Laboratory Inc., and AGILENT® Technologies. In some embodiments of the kits described herein, at least one of the labelled antibodies comprises an enzyme-labelled antibody. The anti-mycolactone that is bound and captured by the immobilized mycolactone on the solid support (e.g. microtiter plate wells) is identified by adding a chromogenic substrate for the enzyme conjugated to the anti-mycolactone antibody, e.g. anti-human IgG, and color production detected by an optical device such as an ELISA plate reader. In some embodiments, the kits described herein further comprise standards of known amounts of the mycolactone or fragments thereof. In some embodiments, the kits described herein further comprise reference values of the levels of anti-mycolactone antibodies. The reference values are average levels of anti-mycolactone antibodies in samples from a population of healthy individuals. Reference values can be provided as numerical values, or as standards of known amounts or titer of anti-mycolactone antibodies presented in pg/ml\hat{ }g/ml. In some embodiments, the kits described herein further comprise at least one sample collection container for sample collection. Collection devices and container include but are not limited to syringes, lancets, BD VACUTAINER® blood collection tubes. In some embodiments, the kits described herein further comprise instructions for using the kit and interpretation of results.

The invention will be further illustrated by the following figures and examples. However, these examples and figures should not be interpreted in any way as limiting the scope of the present invention.

### FIGURES

FIG. 1: Detection of cutaneous IgG binding to mycolactone. Each well of an ELISA Maxisorp plate was coated with 3 ng mycolactone, and the immunoglobulin concentrations of cutaneous tissues samples were normalized before their addition to the plate. Antibodies binding to mycolactone on the plate were recognized by HRP-conjugated secondary antibodies. A) Kinetics of the recognition of the mycolactone present in cutaneous tissue by cutaneous IgG (n=5 for each mouse strain). B) Kinetics of cutaneous IgG1, IgG2a, IgG3 and IgG2b recognition of mycolactone in cutaneous tissues from FVB/N and C57Bl/6 mice (n=3 to 5 mice per mouse strain) at each time point in clinical M. ulcerans infection (healthy, redness, ulcer, necrosis or healing). The detection limit was an absorbance of 0.1. The histograms show the means±SD. * p<0.5 (Mann-Whitney U test).

FIG. 2: Neutralization of mycolactone by cutaneous immunoglobulin from FVB/N mice infected with M. ulcerans. CD4+ lymphocytes were stimulated with PMA/ionomycin. Mycolactone was used at a concentration of 4 ng/mL. Immunoglobulins purified from the skin of A) FVB/N mice or B) C57Bl/6 mice infected with M. ulcerans were used at a ratio of 10 immunoglobulin molecules per molecule of mycolactone. Left panel: IL-2 quantification, Right panel: IFNγ quantification. The data shown correspond to one experiment performed in triplicate (mean±SD).

FIG. 3: Detection of cutaneous immunoglobulins (IgG) binding to mycolactone in the lesions of patients diagnosed with Buruli ulcer. Wells were coated with mycolactone, and antibodies binding to mycolactone in the plate were recognized by HRP-conjugated secondary antibodies. Biopsy specimens from patients with suspected and/or PCR-confirmed Buruli ulcer were provided by the CDTUB of Pobè (Benin). The detection limit was an absorbance of 0.150. ** p<0.05 (Mann Whitney U test). Buruli ulcer group n=15, control group n=19.

### EXAMPLE

Material & Methods

Ethics Statement for Animal Experiments and Use of Human Tissues

All animal experiments were performed in accordance with national guidelines (articles R214-87 to R214-90 of the French “rural code”) and European guidelines (directive 2010/63/EU of the European Parliament and of the Council of Sep. 22, 2010 on the protection of animal used for scientific purposes). All protocols were approved by the ethics committee of the Pays de la Loire region, under protocol no. APAFIS8904. Mice were housed in specific pathogen-free conditions in the animal house of the Angers University Hospital, France (agreement A 49 007 002). The use of biopsy samples from patients for research purposes was approved by the research committee of government of Benin (Ministry of Health, Republic of Benin, agreement number 2893).

M. ulcerans Strain and Inoculation

Mycobacterium ulcerans strain 01G897 was originally isolated from patients from French Guiana (18). A bacterial suspension was prepared, as previously described (19, 8), and its concentration was adjusted to 1×104 acid-fast bacilli/mL for inoculation (50 μL) into the tails of six-week-old female consanguineous C57Bl/6 and FVB/N mice (Janvier, Le Genest Saint Isle, France).

Mycolactone

The Malaysian M. ulcerans 1615 strain was cultured on solid 7H10 medium supplemented with 10% OADC (oleic acid, dextrose, catalase; Difco, Becton-Dickinson) at 30° C. for 45 days. Mycolactone A/B was then purified from whole bacteria, as previously described (20).

Mycolactone was diluted to a concentration of 3 mg/mL in absolute ethanol and stored in the dark, in amber glass tubes, at −20° C.

Mouse Tissue Preparation and Serum Sampling

Skin samples from clinically infected mice (displaying redness, edema, ulcer, necrosis or healing) were excised (we removed one centimeter of cutaneous tissue from around the lesion) and crushed in PBS supplemented with Complete EDTA-free cocktail (Roche), with a TissueRuptor (Qiagen). The resulting suspensions were centrifuged at 3,500×g for 10 minutes at 4° C. The supernatants were stored at −80° C. Blood samples were collected by retro-orbital puncture at each clinical stage of infection, before tail excision. The blood was centrifuged at 1,500×g for 10 minutes to isolate serum, which was recovered and stored at −80° C.

Preparation of Human Biopsy Tissues

Skin biopsy specimens were provided by the CDTLUB of Pobé (Benin). The biopsy specimens were crushed in PBS supplemented with protease inhibitors (Complete EDTA-free cocktail, Roche), with a TissueRuptor (Qiagen), as described for mouse tissues. The resulting suspensions were centrifuged at 3,500×g for 10 minutes at 4° C. The resulting supernatants were stored at −80° C.

Quantitative PCR Analysis

Tail skins from infected mice were excised and immediately placed into RNAlater (Qiagen) and stored at −20° C. Skin tissues were crushed and homogenized with a TissueRuptor (Qiagen) and total RNA was then purified with the RNeasy fibrous tissue midi kit (Qiagen). The first-strand cDNA was synthesized from 750 ng RNA with the M-MLV reverse transcriptase (Invitrogen). Quantitative PCR was performed to quantify the levels of IgM, IgA and IgG mRNA. Specific gene expression was calculated by the relative expression method (using actin as the calibrator). The sequences of the primers and probes used are provided in Supplementary table 1.

Quantification of Proteins and Immunoglobulins

Total protein levels were determined with a colorimetric assay (Protein Assay Dye Reagent), according to the manufacturer's instructions, with Dye Reagent Concentrate Refill (Bio-Rad 5000006) and a standard curve (bovine gamma globulin, kit 1, 50000001, Bio-Rad). Mouse tissue samples were normalized to identical protein concentrations before testing. IgA, IgM and IgG were quantified in crushed tissue samples and mouse sera by ELISA kit from eBioscience, according to the manufacturer's recommendations (Mouse IgA Ready-SET-Go, 88-50450; Mouse IgM Ready-SET-Go, 88-50470 and Mouse IgG Ready-SET-Go, 88-50400).

Immunoglobulins Purification

Tail skins from infected mice were excised and crushed with metallic beads and a TissueRuptor (Qiagen) in an equal volume of PBS supplemented with proteases inhibitor (Complete EDTA-free cocktail Roche) and protein A/G IgG Binding Buffer (Thermo Fisher Scientific). The resulting suspensions were centrifuged at 8,000×g for 45 minutes at 4° C. Ig were then purified with a Nab™ protein A/G spin column (Thermo Fisher Scientific) followed by a Nab™ protein L spin column (Thermo Fisher Scientific), according to the manufacturer's instructions.

Detection of Antibodies Directed Against M. ulcerans Lysate by ELISA

M. ulcerans lysate was prepared as previously described (8). M. ulcerans lysate (0.5 μg), diluted in 100 μL of sodium bicarbonate buffer (50 mM; pH 9.6), was immobilized in 96-well ELISA plates (Thermo Fisher Scientific®, Nunc-Immuno™ Plates, Maxisorp 456537) by overnight incubation at 4° C. The coated plates were washed four times with 0.05% Tween 20 in PBS and were then saturated by incubation with 5% skim milk powder in PBS for 2 h at room temperature. The plates were washed a further four times and were then incubated with crushed tissue samples with normalized protein contents in 1% skim milk powder in PBS for 2 h at room temperature. After four washes, antibodies directed against M. ulcerans lysate were detected with horseradish peroxidase (HRP)-conjugated secondary antibodies diluted 1:500 in 1% skim milk powder in PBS. The plates were incubated with the secondary antibodies (Supplementary table 1) for 1 h at room temperature and were then washed four times. The SureBlue™ TMB Microwell Peroxidase Substrate (KPL) was used for secondary antibody detection, and 1 M H2SO4 was added to stop the reaction. Absorbance was measured at λ=450 nm (with a reference at λ=570 nm) (Thermo Fisher Scientific®, Multiskan Ascent); results are expressed in optical density units.

Detection of Mycolactone-Binding Antibodies

ELISA was performed to detect antibodies directed against mycolactone present in human tissues, mouse cutaneous tissues and mouse sera. Mycolactone A/B (3 ng) in absolute ethanol was immobilized in 96-well plates (Thermo Fisher Scientific®, Nunc-Immuno™ Plates, Maxisorp 456537), by evaporating off the ethanol; coated plates were stored at −20° C. in dark. They were incubated overnight at 4° C. with 5% skim milk in PBS. The protein concentrations of mouse tissue samples were normalized, mouse serum samples were 10-fold diluted and human samples diluted two-fold. The plates were washed three times in 0.05% Tween 20 in PBS and were then incubated with diluted samples for 2 h at room temperature. The plates were washed four times and incubated with HRP-conjugated secondary antibodies (Goat anti-mouse IgG1, Goat anti-mouse IgG2a, Goat anti-mouse IgG2b, Goat anti-mouse IgG3, Goat anti-mouse IgG/IgA/IgM (H+L) (diluted 1:1000 in 1% skimmed milk in PBS for 2 h at room temperature (supplementary table 1). Bound antibodies were revealed by using the SureBlue™ TMB Microwell Peroxidase Substrate (KPL) was used for secondary antibody detection, and 1 M H2SO4 was added to stop the reaction. Absorbance was measured at 450 nm and is expressed in optical density units.

Neutralizing Activity Assay

CD4+ T cells were isolated from the spleens of C57Bl/6 mice by magnetic sorting (MACS technology kit 130-104-454), according to the manufacturer's instructions (Miltenyi Biotec). The purity of CD4+ T-cell preparations was determined by flow cytometry, with PE-conjugated anti-CD4 monoclonal antibody (eBioscience) and FITC-conjugated anti-CD3E monoclonal antibody (eBioscience). CD4+ T cells (200,000 cells/well; 100 μL per well) were used to seed 96-well plates. Purified IgG from infected tissue was diluted in RPMI 1640 (Lonza) supplemented with 10% FCS (Eurobio), 2 mM glutamine, 10 U/mL streptomycin, and 100 U/mL penicillin (Lonza). In some conditions, these were mixed with 8 ng/mL mycolactone A/B in a 10:1 ratio in a tube and incubated at 37° C. with continuous stirring for 45 minutes. Fetal calf serum was added to the preparation at a final concentration of 10%, followed by 10 ng/mL PMA and 1 nM ionomycin. 100 μL per well of this preparation was then added to CD4+ T cells. Cells were incubated for 6 h at 37° C. under an atmosphere containing 5% CO2, and supernatants were collected and stored at −20° C. IL-2 and IFN-γ were quantified by ELISA (eBioscience), according to the manufacturer's instructions (Mouse IL-2 Ready-SET-Go: 88-7024 and Mouse IFN-γ Ready-SET-Go: 88-7314 respectively).

Immune Cell Isolation and Flow Cytometry Analysis

Tail skin was excised from three mice. Tissues were digested with Multi Tissue Dissociation kit 1 from Miltenyi Biotec (reference 130-110-201) according to the manufacturer's instructions. A four-color staining method was used to identify B-cell subsets: CD45+, CD19+, B220+ and CD138+ cells were labeled with APC cyanidine7 anti-CD45 (BD Biosciences), phycoerythrin anti-CD19 (BD Biosciences), [phycoerythrin-cyanidine 7] anti-CD45R/B220 (BD Biosciences) and BB515anti-CD138 (BD Biosciences) antibodies; dead cells were excluded from the analysis by staining with 7AAD (Miltenyi Biotec). Flow cytometry analysis was performed on a MACSQuant analyzer. Results were analyzed with FlowLogic software.

Immunohistology

Tails from infected mice were excised and immediately fixed by incubation in 4% paraformaldehyde for 24 h. Tissues were then embedded in paraffin, and cut into 5 mm-thick sections. Hematoxylin-phloxine-saffron (HPS) staining was performed according to the manufacturer's protocol. Immunohistochemical staining was performed with an anti-CD138 polyclonal antibody (Thermo Fisher Scientific ref. #36-2900) diluted 1:250 according to the manufacturer's protocol.

Statistical Analysis

The data, presented as means and SE (standard error), were analyzed with GraphPad Prism 5.0 software (GraphPad Software, San Diego, Calif., USA). Different clinical stages in each mouse strain were compared in Kruskal-Wallis tests with Dunn's Multiple Comparison test. FVB/N and C57Bl/6 mice were compared, at each clinical stage, in Mann-Whitney U tests. Buruli ulcer patients and controls were analyzed in Mann-Whitney U tests.

Results

Production of Skin Immunoglobulins During M. ulcerans Infection

We investigated the local humoral response during M. ulcerans infection, including the spontaneous healing process, by evaluating immunoglobulin gene expression through analyses of mRNA levels in cutaneous tissue samples. The FVB/N mouse model (which displays spontaneous healing) was compared with the C57Bl/6 mouse (which displays no such healing). Relative mRNA levels for IgM, IgA and IgG remained stable in both models during early stages of infection (Data not shown). However, the levels of mRNA for all three immunoglobulin isotypes increased significantly in FVB/N mice at the ulcerative stage (day 45), reaching a peak during the healing stage (day 75). The levels of mRNA encoding these three isotypes were significantly higher (p-value<0.05 for IgM and IgG and p-value<0.01 for IgA, Mann-Whitney U test) in FVB/N mice at the healing stage than in C57Bl/6 mice at the necrotic stage. We characterized the immunoglobulin profiles of infected tissues by estimating IgM, IgA and IgG concentrations by ELISA. Consistent with the mRNA data, total immunoglobulin levels increased in the cutaneous tissues of FVB/N mice throughout M. ulcerans infection (Data not shown). However, despite the absence of clear changes in local levels of immunoglobulin mRNA in C57B1l/6 mice, we observed an increase in tissue Ig concentration throughout M. ulcerans infection in these mice (Data not shown). Differences in IgM and IgG concentrations were detected between the two mouse strains. Indeed, IgG levels in FVB/N mice were twice higher than in C57Bl/6 mice (p-value <0.01, Mann-Whitney U test) during both the redness and ulcerative stages, and were 1.6 times higher (p-value <0.05, Mann-Whitney U test) during the healing process than during the necrotic stage, whereas IgM levels were higher in C57Bl/6 mice from the ulcerative stage (2.7 times higher, p-value <0.01, Mann-Whitney U test) until necrosis (7.8 times higher, p-value <0.01, Mann-Whitney U test). These differences, evident from the earliest stages, could reflect elevated circulating immunoglobulins. However, no significant differences in the levels of these immunoglobulins were detected between the serum samples of the two mouse strains during the early stages of infection (Data not shown). Thus, the course of M. ulcerans infection is associated with a local humoral response, with the following immunoglobulin profiles: IgM>IgG>IgA in C57Bl/6 mice and IgG>IgM>IgA in FVB/N mice. There thus seems to be a specific humoral immunity signature of the healing process, in which IgG predominates.

Recognition of M. ulcerans Lysate by Iocal IgG Antibodies

We investigated the specificity of this local immunoglobulin production, by evaluating their capacity to recognize the whole M. ulcerans lysate in an ELISA assay. IgG was the only isotype of immunoglobulin able to bind M. ulcerans lysate components (Data not shown). This recognition became stronger with successive stages of infection in both FVB/N and C57Bl/6 mice, and was maximal during the healing and necrosis stages (p-value <0.01, Mann-Whitney Test). We then evaluated the ability of IgG subclasses to recognize M. ulcerans components (Data not shown). IgG1 bind at high levels to the M. ulcerans lysate components in both mouse models. IgG1 binding was detected early in the redness stage in FVB/N mice and later, at the edema stage in C57Bl/6 mice (p-value <0.01 for the difference between the two mouse models at the redness stage, Mann-Whitney U test). By contrast, IgG3 recognized M. ulcerans lysate components very weakly, but similarly in the two mouse strains. The major difference between FVB/N and C57Bl/6 mouse concerned the IgG2a/b subclasses. IgG2a recognized M. ulcerans components at all stages of infection (from the ulcerative to the healing stage) in FVB/N mice, but seemed to be absent in C57Bl/6 mice. The last subclass, IgG2b, recognized M. ulcerans lysate significantly more strongly in C57Bl/6 mice, from the ulcerative to the necrotic stage, than in FVB/N mice at equivalent stages (p-value <0.05, Mann-Whitney U test). In conclusion, local humoral responses differed between FVB/N mice (healing model) and C57Bl/6 mice (not able to heal). This difference mostly concerned two specific immunoglobulin subclasses: (i) IgG2a, which was produced only in the healing model, and (ii) IgG2b, which recognized M. ulcerans components strongly only in the C57Bl/6 model. These results suggest a potential role for IgG2a in controlling M. ulcerans infection in the FVB/N healing model.

Recognition of Mycolactone by Local Skin Antibodies

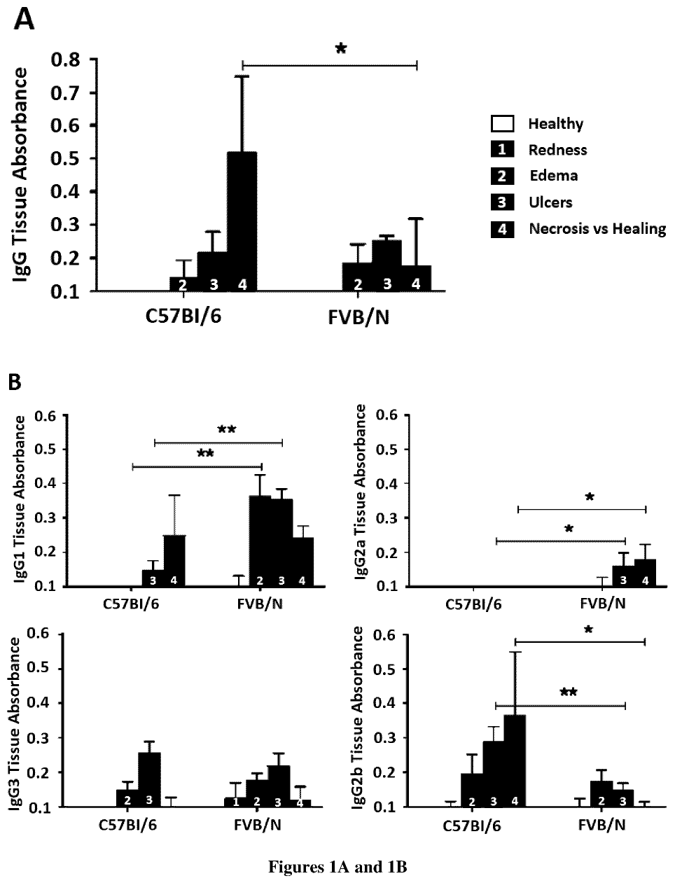

The ability of FVB/N mice to heal spontaneously after M. ulcerans infection could be explained, in part, by the production of antibodies recognizing the bacterial toxin, mycolactone. We investigated the specificity of these local antibodies for mycolactone in skin tissues by performing ELISA (FIGS. 1A and 1B). IgM produced locally in both mouse strains were unable to recognize mycolactone. IgA, which is known to protect tissues (ie. epithelial cells) against bacterial toxins (Janeway 2001), bound mycolactone at low levels only during the ulcerative stage in FVB/N mice. As observed for M. ulcerans lysate, IgG recognized mycolactone at high levels in both mouse models throughout infection. The level of mycolactone recognizing by IgG was significantly higher in C57Bl/6 mice at the necrotic stage than in FVB/N mice at the healing stage (p-value <0.05, Mann-Whitney U test). We then analyzed IgG subtypes at each stage of infection. From the edema stage to the ulcerative stage, IgG1 appeared to be the principal subclass of mycolactone-binding antibody in FVB/N mice, whereas IgG2b seemed to be present exclusively in C57Bl/6 mice (p-value <0.01 for IgG1 in FVB/N versus C57Bl/6 mice, p-value <0.01 for IgG2b in the ulcerative stage in C57Bl/6 mice relative to FVB/N mice, Mann-Whitney U test). During the last stage of infection (necrosis vs. healing), IgG1 antibody seemed to be the principal isotype at work in the FVB/N model, whereas IgG2b remained the major subclass of antibody binding the toxin in C57Bl/6 mice (p-value <0.05 versus FVB/N mice, Mann-Whitney U test). The mycolactone-binding profile of IgG3 was similar in both mouse models. Finally, as for the recognition of M. ulcerans lysate, the major difference concerned the IgG2a subclass, which appears to be specific to FVB/N mice. This subclass bound the toxin from the ulcerative stage to the healing stage. The lack of IgG2a detection in assays performed on C57Bl/6 mice may be explained by a mutation of the IgG2a gene in these mice (27). For exclusion of a strain-specific phenotype and confirmation of the importance of this protein in the healing process and in mycolactone binding, we performed assays in another mouse model displaying no spontaneous healing (BALB/c mice) in which the IgG2a gene is present and functional. We found that the IgG2a antibody produced by BALB/c mice did not recognize mycolactone (Data not shown). In parallel, the systemic humoral recognition of mycolactone was assessed in FVB/N and C57Bl/6 mice, but no systemic antibodies binding the toxin were detected (data not shown). Taken together, these results reveal different mycolactone recognition profiles between mouse models of healing and necrotic infection. IgG2b appears as the principal subclass able to recognize M. ulcerans toxin in C57Bl/6 mice, but appears to be unable to control infection efficiently. By contrast, IgG2a was specific to the spontaneous healing model (FVB/N mice) and may be involved in the control of M. ulcerans infection associated with spontaneous healing.

Neutralization of Mycolactone by Antibodies Present in the Skin

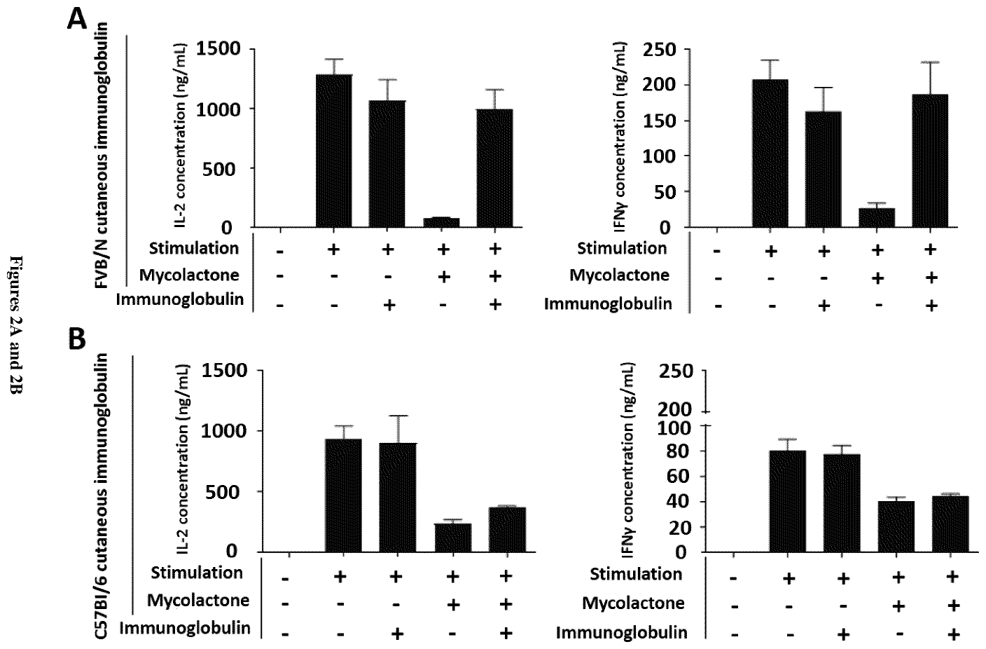

In this context, we assessed whether these antibodies could not only recognized but also neutralized mycolactone, by evaluating cytokines production by T lymphocytes as an indicator of the immunomodulatory property of mycolactone. To this end, we compared the IL-2 and IFNγ production by T cells after the stimulation of these cells and their incubation with mycolactone alone or mycolactone previously incubated with IgG purified from the skin of FVB/N and C57Bl/6 mice. Contact between T cells and the toxin greatly decreased IL-2 and IFNγ production as compared to the positive control (T cells only stimulated) in both mouse models (FIGS. 2A and 2B). By contrast, the production of these two cytokines was similar to the positive control for T cells in contact with mycolactone previously incubated with IgG purified from the skin of FVB/N mice, but not for T cells in contact with mycolactone previously incubated with IgG purified from C57Bl/6 mice. Taken together, these results show that IgG produced by FVB/N mice skin during the healing stage can neutralize the toxin of M. ulcerans. This neutralization may be involved in the healing process.

Local Emergence of Antibody-Producing Cells During M. ulcerans Infection

Consistent with the rational of the establishment of a local humoral immune response during spontaneous healing, we tried to demonstrate the physiological relevance of this process by investigating the presence of antibody-producing B cells in vivo. Using four-color staining, we analyzed B cell populations from FVB/N mice, and identified three populations: (P1) cells with a CD45+, CD19+, B220+ phenotype corresponding to B cells, (P2) CD45+, CD19+, B220int cells corresponding to plasmablasts, and (P3) CD45+, CD19−, B220int, CD138+ cells, corresponding to the murine markers of the last stage of B-cell maturation, plasma cells (Tellier & Nutt. 2017). The total number of lymphoid cells (CD45+) increased 37-fold as compare to the control skin during the spontaneous healing process (Data not shown). Consequently, the total number of B cells increased, but the proportions of the various subsets remained constant, except for the plasma cell subset, which increased in proportion from 0.07 to 0.54% of total lymphoid cells during the first few steps of the spontaneous healing process (Day 55), whereas no such increase was observed in uninfected skin (control) (Data not shown). The presence of this specific B-cell subtype was confirmed by histological analysis (Data not shown). The proportion of B1-like B cells (CD45+, CD19+, B220int, CD43+), a specific subset which has been shown to produce antibodies specifically in the skin (28) increased during spontaneous healing, reaching 0.07% of total lymphoid cells (Data not shown). The proportion of these antibody-producing cells decreased when the lesion appears to be completely healed (D75), but remained higher than in control skin. Finally, we have shown that antibody-producing cells strongly increase in the skin during the spontaneous healing process, supporting the role of the local humoral response in this phenomenon, through the production of anti-mycolactone antibodies.

Recognition of Mycolactone by Local IgG Purified from Skin Biopsy Specimens from Buruli Ulcer Patients

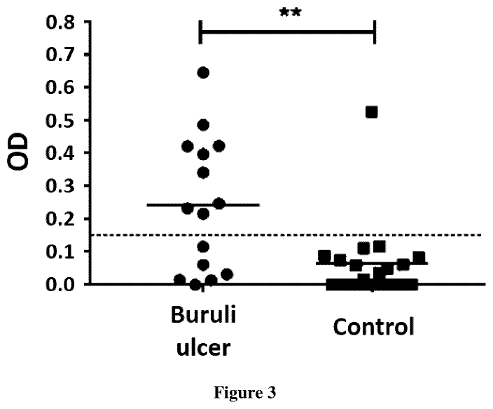

In addition to this detailed characterization of anti-mycolactone immunoglobulins in mice, we also assessed the levels of these IgGs in Buruli ulcer patients. We used ELISA to detect anti-mycolactone antibodies in skin samples from patients (either diagnosed or not diagnosed as Buruli ulcer patients) provided by the CDTUB of Pobé (Benin). In 73% of PCR-confirmed Buruli ulcer patients, the most severe form of the disease had been diagnosed: an ulcerative lesion (which may be associated with other forms, such as edema or plaques). Mycolactone was recognized by local antibodies recovered from the lesions of 60% of patients with PCR-confirmed Buruli ulcer (9/15) (all presenting an ulcer), whereas mycolactone was not detected in the biopsy specimens of all but one of the control patients (not diagnosed with Buruli ulcer; p-value<0.05, Mann-Whitney U test; FIG. 3). This particular patient has since then developed squamous cell carcinoma, a disease that is known to appear in some cases after Buruli ulcer lesions (29, 30). We cannot, therefore, exclude the possibility that this patient may have already had a lesion due to M. ulcerans infection. Finally, there may be several reasons for the lack of mycolactone-binding IgG detection in 40% of patients with PCR-confirmed Buruli ulcer (6/15) such as for instance the type of lesion, the sampling site (distance from the site of infection), and the treatment duration. Finally, we demonstrate here, for the first time to our knowledge, that the human organism can generate an effective humoral response involving the production of antibodies able to recognize mycolactone during M. ulcerans infection.

Discussion

Buruli ulcer is a neglected tropical disease and remains the third most common mycobacterial disease worldwide. This debilitating skin disease is caused by M. ulcerans, which produces a lipid-like toxin, mycolactone, the main virulence factor of the bacillus. Without treatment, lesions can escalate into chronic skin ulcers. However, these severe lesions can spontaneous heal, as observed in 5% of patients cases, suggesting that the host may be able to develop strategies for counteracting the effects of M. ulcerans. Despite the development of animal models, the mechanisms of the spontaneous healing process remain unclear. We previously showed (i) the absence of a systemic immune cell response signature and (ii) a weak involvement of the local cellular immune response in the switch from acute to chronic infection (8). These results highlight the local consequences of the disease, as observed with other approaches. Indeed, recent histological studies have shown that B cells accumulate in clusters around the site of M. ulcerans infection (10). B-cell infiltrates have been observed in chronic inflammatory skin conditions, including cutaneous leishmaniosis and atopic dermatitis (33, 34). The debate about the pro- or anti-inflammatory role of skin B cells is still ongoing, yet these cells have been shown to be involved in the resolution of skin inflammation in a mouse model of psoriasis-like inflammation (35). Furthermore, in addition to producing antibodies locally, B cells have been shown to play a role in the process of wound healing (36).

In this context, we used our mouse model of spontaneous healing to investigate the humoral response at all stages of M. ulcerans infection, including spontaneous healing. We demonstrated the presence of antibody-producing B cells during infection and their increase in number during infection, peaking during the early stages of spontaneous healing (the transition from ulceration to healing). We detected immunoglobulins able to recognize the lipid toxin of M. ulcerans, mycolactone, throughout all stages of infection. Interestingly, these immunoglobulins were found in the skin of infected mice, but not in their sera. We also provide the first demonstration of the presence of these immunoglobulins in biopsy specimens from Buruli ulcer patients. No anti-mycolactone antibodies have ever been detected in serum from patients. Collectively, these results highlight the existence of a distinct humoral signature in response to M. ulcerans infection, with the skin-specific production of antibodies against mycolactone.

The specific local production of immunoglobulins able to recognize M. ulcerans components, including mycolactone in particular, may contribute to the control of infection observed during spontaneous healing. We investigated this possibility, by evaluating the ability of IgG subclasses to recognize M. ulcerans components. We tagged, for the first time, a specific subclass of immunoglobulin, IgG2a, which is known to diffuse readily in the skin (37). This subclass reported as highly effective on neutralizing bacterial exotoxins, such as diphtheria toxin, enterotoxin B or Bacillus anthracis-associated toxin (38), was produced only in the spontaneous healing model and seems to be the signature of this model in terms of mycolactone recognition. Our results also showed that IgG isolated from the skin of FVB/N mice that was able to recognize M. ulcerans components neutralized similarly the toxic activity of mycolactone. No other mycolactone-neutralizing antibodies have been identified in other mouse models.

The neutralization of bacterial toxins is an important part of the humoral immune response to bacterial infections (38). Our results suggest therefore that mycolactone neutralization may be the key to the spontaneous healing process of Buruli ulcer. Indeed, this phenomenon may block mycolactone activity in various ways: it would probably be more difficult for mycolactone linked to an antibody to gain access to or inhibit known intracellular targets, such as the Sec61 translocon (i) if the antibody-mycolactone complex cannot cross membranes or (ii) if the antibody hampers the fixation of the mycolactone. It is therefore reasonable to assume that (iii) neutralizing antibodies may help to eliminate the toxin by targeting it to phagocytes cells.

The existence of anti-mycolactone antibodies opens up exciting new perspectives for innovations in diagnosis, treatment and vaccine development responding to the scientific challenge issued by the World Health Organization. Indeed, there is currently no simple diagnostic tool suitable for use in the rural areas of developing countries. This situation is particularly regrettable, as the early stages of Buruli ulcer can be treated locally, whereas the treatment of later stages requires extensive surgery in larger hospitals, with longer periods of hospitalization, at much greater expense. The development of a new diagnostic tool, such as a test based on monoclonal antibody production, might be more appropriate for these endemic regions.

### REFERENCES

Throughout this application, various references describe the state of the art to which this invention pertains. The disclosures of these references are hereby incorporated by reference into the present disclosure.
1. F. Portaels, M. T. Silva, W. M. Meyers, Buruli ulcer. Clin Dermatol 27, 291-305 (2009).
2. E. Marion et al., Mycobacterial toxin induces analgesia in buruli ulcer by targeting the angiotensin pathways. Cell 157, 1565-1576 (2014).
3. C. Demangel, T. P. Stinear, S. T. Cole, Buruli ulcer: reductive evolution enhances pathogenicity of Mycobacterium ulcerans. Nat Rev Microbiol 7, 50-60 (2009).
4. L. Marsollier et al., Colonization of the salivary glands of Naucoris cimicoides by Mycobacterium ulcerans requires host plasmatocytes and a macrolide toxin, mycolactone. Cell Microbiol 7, 935-943 (2005).
5. L. Mosi et al., Mycobacterium ulcerans causes minimal pathogenesis and colonization in medaka (Oryzias latipes): an experimental fish model of disease transmission. Microbes Infect 14, 719-729 (2012).
6. R. E. Simmonds, F. V. Lali, T. Smallie, P. L. Small, B. M. Foxwell, Mycolactone inhibits monocyte cytokine production by a posttranscriptional mechanism. J Immunol 182, 2194-2202 (2009).
7. A. G. Fraga et al., Mycobacterium ulcerans triggers T-cell immunity followed by local and regional but not systemic immunosuppression. Infect Immun 79, 421-430 (2011).
8. E. Marion et al., FVB/N Mice Spontaneously Heal Ulcerative Lesions Induced by Mycobacterium ulcerans and Switch M. ulcerans into a Low Mycolactone Producer. J Immunol 196, 2690-2698 (2016).
9. K. M. Dobos, E. A. Spotts, B. J. Marston, C. R. Horsburgh, Jr., C. H. King, Serologic response to culture filtrate antigens of Mycobacterium ulcerans during Buruli ulcer disease. Emerg Infect Dis 6, 158-164 (2000).
10. M. T. Ruf, C. Steffen, M. Bolz, P. Schmid, G. Pluschke, Infiltrating leukocytes surround early Buruli ulcer lesions, but are unable to reach the mycolactone producing mycobacteria. Virulence 8, 1918-1926 (2017).
11. K. Asiedu, S. Etuaful, Socioeconomic implications of Buruli ulcer in Ghana: a three-year review. Am J Trop Med Hyg 59, 1015-1022 (1998).
12. Q. B. Vincent et al., Clinical epidemiology of laboratory-confirmed Buruli ulcer in Benin: a cohort study. The Lancet. Global health 2, e422-430 (2014).
13. E. Marion et al., Clinical Features of Spontaneous Partial Healing During Mycobacterium ulcerans Infection. Open Forum Infect Dis 3, ofw013 (2016).
14. D. P. O'Brien et al., Spontaneous healing of Mycobacterium ulcerans disease in Australian patients. PLoS Negl Trop Dis 13, e0007178 (2019).
15. I. U. Egbuniwe, S. N. Karagiannis, F. O. Nestle, K. E. Lacy, Revisiting the role of B cells in skin immune surveillance. Trends Immunol 36, 102-111 (2015).
16. S. A. Geherin et al., The skin, a novel niche for recirculating B cells. J Immunol 188, 6027-6035 (2012).
17. S. A. Geherin, M. H. Lee, R. P. Wilson, G. F. Debes, Ovine skin-recirculating gammadelta T cells express IFN-gamma and IL-17 and exit tissue independently of CCR7. Vet Immunol Immunopathol 155, 87-97 (2013).
18. P. L. De Gentile et al., [Cutaneous ulcer from Mycobacterium ulcerans. Apropos of 1 case in French Guiana]. Bull Soc Pathol Exot 85, 212-214 (1992).
19. L. Marsollier et al., Impact of Mycobacterium ulcerans biofilm on transmissibility to ecological niches and Buruli ulcer pathogenesis. PLoS Pathog 3, e62 (2007).
20. E. Marion et al., Photodegradation of the Mycobacterium ulcerans toxin, mycolactones: considerations for handling and storage. PLoS One 7, e33600 (2012).
21. P. Du, W. A. Kibbe, S. M. Lin, lumi: a pipeline for processing Illumina microarray.

Bioinformatics (Oxford, England) 24, 1547-1548 (2008).
22. C. Schurmann et al., Analyzing illumina gene expression microarray data from different tissues: methodological aspects of data analysis in the metaxpress consortium. PLoS One 7, e50938 (2012).
23. M. Lek et al., Analysis of protein-coding genetic variation in 60,706 humans. Nature 536, 285-291 (2016).
24. M. E. Ritchie et al., limma powers differential expression analyses for RNA-sequencing and microarray studies. Nucleic acids research 43, e47 (2015).
25. G. Bindea et al., ClueGO: a Cytoscape plug-in to decipher functionally grouped gene ontology and pathway annotation networks. Bioinformatics (Oxford, England) 25, 1091-1093 (2009).
26. Y. Hochberg, Y. Benjamini, More powerful procedures for multiple significance testing. Statistics in medicine 9, 811-818 (1990).
27. R. M. Martin, J. L. Brady, A. M. Lew, The need for IgG2c specific antiserum when isotyping antibodies from C57BL/6 and NOD mice. Journal of immunological methods 212, 187-192 (1998).
28. S. A. Geherin et al., IL-10+ Innate-like B Cells Are Part of the Skin Immune System and Require alpha4beta1 Integrin To Migrate between the Peritoneum and Inflamed Skin. J Immunol 196, 2514-2525 (2016).
29. E. Minutilli et al., Squamous cell carcinoma secondary to buruli ulcer. Dermatologic surgery: official publication for American Society for Dermatologic Surgery [et al.] 33, 872-875 (2007).
30. K. Kassi et al., Squamous cell carcinoma secondary to Buruli ulcer: a clinical case report in a young girl. Bacteriologia, virusologia, parazitologia, epidemiologia (Bucharest, Romania: 1990) 55, 25-28 (2010).
31. Y. Benjamini, Y. Hochberg, Controlling The False Discovery Rate—A Practical And Powerful Approach To Multiple Testing. (1995), vol. 57, pp. 289-300.
32. J. Manry et al., Deciphering the genetic control of gene expression following Mycobacterium leprae antigen stimulation. PLoS genetics 13, e1006952 (2017).
33. B. Geiger et al., Resolving lesions in human cutaneous leishmaniasis predominantly harbour chemokine receptor CXCR3-positive T helper 1/T cytotoxic type 1 cells. Br J Dermatol 162, 870-874 (2010).
34. D. Simon, S. Hosli, G. Kostylina, N. Yawalkar, H. U. Simon, Anti-CD20 (rituximab) treatment improves atopic eczema. The Journal of allergy and clinical immunology 121, 122-128 (2008).
35. K. Yanaba et al., Regulatory B cells suppress imiquimod-induced, psoriasis-like skin inflammation. J Leukoc Biol 94, 563-573 (2013).
36. N. Nishio, S. Ito, H. Suzuki, K. Isobe, Antibodies to wounded tissue enhance cutaneous wound healing. Immunology 128, 369-380 (2009).
37. C. A. Janeway, Jr., How the immune system protects the host from infection.

Microbes Infect 3, 1167-1171 (2001).
38. N. Abboud et al., A requirement for FcgammaR in antibody-mediated bacterial toxin neutralization. J Exp Med 207, 2395-2405 (2010).

1. Method of diagnosing and treating Buruli ulcer in a subject comprising detecting anti-mycolactone immunoglobulin (anti-mycolactone IgG) in a biological sample obtained from said subject, and if anti-mycolactone IgG is detected, then administering one or more antibiotics to the subject.

2. The method of claim 1 wherein the sample is a skin tissue.

3. The method of claim 1 wherein the detection and quantification of anti-mycolactone IgG in the sample is performed by ELISA.

4. The method of claim 1 wherein the detection and quantification of anti-mycolactone IgG is performed with a kit or device comprising at least a mycolactone or fragments thereof; at least one solid support wherein the mycolactone or fragments thereof is deposited on the support and a detection antibody, wherein the detection antibody is specific for the anti-mycolactone IgG in the sample of the subject and the detection antibody produces a detectable signal.
